(19) United States
(12) Patent Application Publication
(10) Pub. No.: US 2021/0315973 A1
(43) Pub. Date: Oct. 14, 2021

(54) COMPOSITIONS AND METHODS USING INTERFERON FOR TREATING VIRAL RESPIRATORY INFECTIONS

(71) Applicant: NOSTRUM PHARMACEUTICALS, LLC, Somerset, NJ (US)

(72) Inventors: Nirmal V. Mulye, Miami, FL (US); Yatindra Prashar, Princeton, NJ (US)

(73) Assignee: NOSTRUM PHARMACEUTICALS, LLC, Somerset, NJ (US)

(21) Appl. No.: 17/224,225

(22) Filed: Apr. 7, 2021

Related U.S. Application Data
(60) Provisional application No. 63/006,921, filed on Apr. 8, 2020.
(60) Provisional application No. 63/028,280, filed on May 21, 2020.
(60) Provisional application No. 63/069,561, filed on Aug. 24, 2020.

(51) Int. Cl. A61K 38/21; A61K 31/4706; A61K 31/56; A61K 45/06

(52) U.S. Cl. CPC A61K 38/21; A61K 45/06; A61K 31/56; A61K 31/4706

(57) ABSTRACT

The present disclosure provides pharmaceutical compositions comprising interferons (IFNs), methods of treating or preventing viral respiratory infections in subjects thereof using IFNs thereby reducing viral respiratory infections, symptoms thereof, inflammation, and minimizing virus propagation. In some embodiments, the pharmaceutical compositions may comprise at least one IFN, an aminoquinoline, a corticosteroid, and one or more pharmaceutically acceptable excipients, carriers, or diluents; and optionally at least one therapeutic agent, and methods of use thereof. Another embodiment may be directed to any of the disclosed pharmaceutical compositions, where the pharmaceutical composition is inhalable.

### CROSS REFERENCE TO RELATED APPLICATIONS

The present application claims priority to U.S. Provisional App. No. 63/069,561, filed Aug. 24, 2020; U.S. Provisional App. No. 63/028,280, filed May 21, 2020; and U.S. Provisional App. No. 63/000,6921, filed Apr. 8, 2020, all of which are hereby incorporated by reference in their entirety.

### FIELD OF DISCLOSURE

The present disclosure is related to compositions useful for the treatment or prophylaxis of viral respiratory infections involving the administration of an interferon. Methods involving these compositions are provided as well.

### BACKGROUND

Viral infections may affect the body's respiratory system comprising the upper and/or lower respiratory tract. These respiratory viral infections may result in the common cold, flu, tonsillitis, laryngitis, sinus infections, bronchiolitis, bronchitis, croup, pneumonia, and the like. Typical symptoms may include coughing, fever, inflammation, fatigue, etc.

Generally viral infections occur when viral particles bind to a receptor on the surface of a host cell membrane, such that the endocytosis occurs and passes the virus into the host cell allowing for the viral genome to be released. The virus uses the host cell for producing proteins in order to replicate its genome, synthesize new viral particles to infect other host cells. Although anti-viral treatments have been used, these are typically broad spectrum anti-virals, agents that inhibit virus replication or cell entry, and immunostimulants. However, these treatments are not necessarily wholly effective or result in adverse reactions or side effects.

Interferons (IFNs) are proteins that are known to interfere with the propagation or replication of viruses inside a cell or cells. There are three subtypes: I, II, and III that initiate signal transduction cascades by binding specific cell surface receptors to initiate immune responses. Human Type I IFNs, structurally similar cytokines (e.g., all lack introns or the length of the protein (161-167 amino acids), and their protein sequence is highly conserved (75-99% amino acid sequence identity) include, but are not limited to: IFN-alpha (IFNα), IFN-beta (IFNβ), IFN-epsilon (IFNε), IFN-kappa (IFNκ), and IFN-omega (IFNω). These type I IFNs bind a ubiquitously expressed heterodimeric transmembrane receptor known as IFN-α receptor (IFNAR) to induce, for example, antiviral effects. The only type II IFN is IFN-gamma (IFNγ) which triggers immune responses to intracellular pathogens. Type III IFNs, including IFN-lambda 1 (IFNλ1) (Interleukin-29 [IL-29]), IFN-λ2 (IL29A), IFNλ3 (IL-28B), and IFNλ4, bind the mucosal surface-abundant receptor complex, IFNλ receptor (IFNLR or also known as IL-28R), which has two subunits: IFNLR1 and IL10R2, to initiate protection against viral infections at mucosal barriers. Binding of these IFNs to their receptors, ultimately leads to the expression of specific genes that result in the activation of the following major events:

Expression of 2′-5′ oligoadenylate synthetase, an enzyme which polymerizes ATP in the cytoplasm into 2′-5′ linked oligomers. These oligomers of ATP activate an endoribonuclease, which then degrades viral RNA.

IFNα and IFNβ also activate a double-stranded RNA dependent protein kinase, which phosphorylates eukaryotic Initiation Factor 2 (eIF2Alpha) inactivating this translation initiator, and thus inhibiting any initiation of translation of viral RNA into protein.

Both IFNα and IFNβ cause induction of Major Histocompatibility Complex (MHC) class 1 proteins on the cell surface, which also carry in them the viral antigens, hence triggering the adaptive immune response where cytotoxic T-cells begin to mount an antigen-specific immune response.

There are very few treatments that actually treat viral infections. Antibiotics are not useful for treating viral infections. In fact, most treatments only treat the symptoms of the viral infection and not the virus itself. However, interferons may be used to treat some viral infections. For example, IFNα may be used to treat Hepatitis B, Hepatitis C, and cancer, while IFNβ may be used to treat Multiple Sclerosis. A typical route of administration of interferons, including IFNα and IFNβ, includes bolus injection, which is a rapid route that administers a large volume of medication over a short period of time. However, there is a need for an effective treatment of viral respiratory infections that is efficiently delivered to the affected areas (e.g., upper and lower respiratory tract) of an infected subject. Although IFNs have antiviral properties, there is a need for an easily administrable, rapid, and effective treatment of viral infections, such as but not limited to coronavirus diseases including SARS-CoV2.

### SUMMARY

In accordance with the foregoing objectives and others, the present disclosure provides pharmaceutical compositions comprising at least one interferon (IFN), optionally inhalable IFNs. Methods of treating or preventing viral respiratory infections in subjects thereof using IFNs to, for example, reduce viral respiratory infections, symptoms associated therewith, reduce inflammation, and minimize virus propagation are also provided. In some embodiments, the pharmaceutical compositions are inhalable pharmaceutical compositions. The compositions, including inhalable compositions, of the disclosure may comprise at least one interferon (IFN), an aminoquinoline (e.g. 4-aminoquinoline) selected from the group consisting of chloroquine, hydroxychloroquine (e.g., hydroxychloroquine sulfate), and one or more pharmaceutically acceptable excipients, carriers, or diluents, and optionally at least one therapeutic agent (e.g., antibiotics, anti-inflammatories, antivirals, anti-parasitic, mucoactive agents, other beneficial or enhancing agents).

Pharmaceutical compositions are provided comprising:
(a) at least one interferon (IFN);
(b) an aminoquinoline selected from chloroquine or hydroxychloroquine or combinations thereof;
(c) an corticosteroid; and
(d) one or more pharmaceutically acceptable excipients, carriers, or diluents.
   These pharmaceutical compositions may further optionally comprise at least one therapeutic agent (e.g., antibiotics, anti-inflammatories, antivirals, anti-parasitics, mucoactive agents, other beneficial or enhancing agents). Other embodiments may provide such compositions where the IFN and/or corticosteroid is inhalable. Furthermore, the pharmaceutical composition may be formulated as an inhalable pharmaceutical composition.

In some embodiments, pharmaceutical compositions are provided comprising:
(a) at least one interferon (IFN);
(b) an aminoquinoline selected from chloroquine or hydroxychloroquine or combinations thereof;
(c) a corticosteroid; and
(d) one or more pharmaceutically acceptable excipients, carriers, or diluents; and
(e) optionally at least one therapeutic agent (e.g., antibiotics, anti-inflammatories, antivirals, anti-parasitics, mucoactive agents, other beneficial or enhancing agents).

Methods of treating a viral respiratory infection in a subject in need thereof are also provided comprising:
(a) administering an interferon (IFN);
(b) administering an aminoquinoline selected from chloroquine, hydroxychloroquine, and combinations thereof, and
(c) administering a corticosteroid (e.g., a glucocorticoid, an inhalable corticosteroid, an oral corticosteroid, an inhalable glucocorticoid), where the IFN and/or the aminoquinoline is inhalable.

In certain implementations, methods of treating COVID-19 in a subject in need thereof are provided which may comprise:
(a) administering an IFNα or IFNβ;
(b) administering azithromycin,
(c) administering hydroxychloroquine; and
(d) administering an corticosteroid,
where one or more of the IFNα or IFNβ, azithromycin, hydroxychloroquine, and corticosteroid is inhalable.

Some embodiments of the disclosure provide methods of treating a viral respiratory infection or methods of treating COVID-19 in a subject in need thereof with any of the compositions disclosed herein, where the composition may optionally be formulated as an inhalable composition.

### BRIEF DESCRIPTION OF THE FIGURES

FIG. 1A is a flow chart of an administration regimen of the present disclosure.

FIG. 1B is a flow chart of an administration regimen of the present disclosure.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, and from 0.1% to 2.5%. It will be understood that the sum of all weight % of individual components will not exceed 100%.

Unless otherwise specified, an indicated percentage is intended to be a weight by weight (w/w) percentage. However, other compositional percentages may be indicated, such as weight/volume (w/v) which, unless otherwise specified, given in g/100 mL. For example, a weight percentage of 0.6% (w/v) is 6 mg/mL.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight. The use of “comprise” is intended to expressly disclose the “consist essentially” and “consist” embodiments.

The term “pharmaceutical composition,” as used herein, represents a composition containing a compound described herein formulated with a pharmaceutically acceptable excipient, carrier, and/or diluent. In some embodiments, the pharmaceutical composition is manufactured or sold with the approval of a governmental regulatory agency as part of a therapeutic regimen for the treatment of disease in a mammal. The active agents disclosed herein combat viral infections, including but not limited to, coronavirus (e.g., HCoV-HKU1, HCoV-OC43, HCoV-NL63, HCoV-229E, MERS-CoV, SARS-CoV, and SARS-CoV-2 or 2019-CoV), influenza virus (e.g., influenza A zoonotic influenza, influenza B), respiratory syncytial virus, parainfluenza virus, adenovirus, and rhinovirus, and may be formulated in pharmaceutical compositions for any route of administration. Non-limiting exemplary routes of administration include oral, intradermal, transdermal (e.g., sustained release formulations), intramuscular, intraperitoneal, intravenous, subcutaneous, epidural, topical, injection, ocular, optic, nasal, nebulization, and inhalation routes. Any other route of administration that is therapeutically effective can be used. It can be used by gene therapy administered to a patient (e.g., via a vector). Furthermore, the proteins according to the disclosure can be administered together with other components of the active agents, such as pharmaceutically acceptable surfactants, excipients, carriers, diluents, and vehicles. For example, for oral administration, the pharmaceutical composition may be formulated in unit dosage form (e.g., a tablet, capsule, caplet, gel cap, lozenge). In certain embodiments, the pharmaceutical composition is formulated as an inhalable formulation, including but not limited to, a spray (e.g., an oral or nasal spray), a dry powder, an aerosol, a liquid, a gas, or atomizable particles or droplets. These formulations may be used in combination with nebulizers, inhalers (e.g., metered-dose inhalers, dry-powder inhalers), gas masks (e.g., SootherMask™; InspiraMask™; available from InspiRx, Inc.), or the like.

The pharmaceutical compositions of the present disclosure are suitable for treating viral respiratory infections resulting in inflammatory conditions of the respiratory system. For example, viral respiratory infections may result in diseases and inflammatory conditions including, but not limited to, common cold, flu, tonsillitis, laryngitis, sinus infections, bronchiolitis, bronchitis, croup, pneumonia, and the like.

As used herein, the phrase “pharmaceutically acceptable” indicates that the specified material is generally safe for ingestion or contact with biologic tissues at the levels employed. Pharmaceutically acceptable is used interchangeably with physiologically compatible.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The compositions of the disclosure can take the form of powders, other formulations (e.g., packaging in lipid-protein vesicles), solutions, suspensions, elixirs, and aerosols. The carrier can be selected from the various oils including those of petroleum, animal, vegetable, or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin, incorporated herein in its entirety. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

The active agents or compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED₅₀ within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Unit dosage forms, also referred to as unitary dosage forms, or pre-metered dosage forms often denote those forms of medication supplied in a manner that does not require further weighing or measuring to provide the dosage (e.g., tablet, capsule, caplet, pre-metered dosage forms used in inhalers or cannisters for nebulizers). For example, a pre-metered dosage form for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gel cap, pre-metered dosage forms for use in nebulizers, inhalers, aerosols, and the like.

The term “effective amount” or “therapeutically effective amount” of an agent (e.g interferons, antibiotics, anti-inflammatories, antivirals, etc.), as used herein, is that amount sufficient to effect beneficial or desired results, such as clinical results, and, as such, an “effective amount” depends upon the context in which it is being applied. In some embodiments, the active agents (e.g., interferons, antibiotics, anti-inflammatories, antivirals, etc.) are administered in an effective amount for the treatment or prophylaxis of a disease, disorder, or condition. In another embodiment, in the context of administering an agent that is one or more interferons either alone or in combination with at least one therapeutic agent (e.g., antibiotics, anti-inflammatories, antivirals), an effective amount of the interferons and therapeutic agent is, for example, an amount sufficient to achieve alleviation or amelioration or prevention or prophylaxis of viral load and/or one or more symptoms or conditions resulting from the common cold, flu, tonsillitis, laryngitis, sinus infections, bronchiolitis, bronchitis, croup, pneumonia, and the like, as compared to the response obtained without administration of the disclosed treatment agents. The viral load (also known as viral burden or viral titer) quantifies the amount of virus or viral particles in an infected subject, which may be tested by nucleic acid amplification based tests (e.g., polymerase chain reaction (PCR), reverse-transcription PCR (RT-PCR), nucleic acid sequence based amplification (NASBA), probe specific amplification methods, signal amplification methods (e.g., branched DNA (bDNA) using either DNA or RNA as targets), including those that may developed in a laboratory setting or commercially available, and/or non-nucleic acid-based tests.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. The term “prevent” or “prophylaxis” as used herein, includes delaying the onset of or progression of a disease or physiological manifestation of disease. The term “treat” includes reducing, diminishing, eliminating, ameliorating, forestalling, slowing the progression of, and/or delaying the onset of a given disease or physiological manifestation thereof.

Typically, the treatment of a condition (e.g., the viral respiratory infections and their conditions or symptoms described herein, e.g., coughing, fever, inflammation, fatigue, etc., is an approach for obtaining beneficial or desired results including clinical results. Inflammation often occurs when tissues are injured by viruses, bacteria, trauma, chemicals, heat, cold, allergens, or any other harmful stimulus. Chemicals including bradykinin, histamine, serotonin and others are released, attracting tissue macrophages and white blood cells to localize in an area to engulf and destroy foreign substances. During this process, chemical mediators such as TNFα are released, giving rise to inflammation. Inflammatory disorders are those in which the inflammation is sustained or chronic. Beneficial or desired results to an inflammatory disease, condition, or disorder can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “subject” refers to any organism to which a composition and/or compound in accordance with the disclosure may be administered, e.g., for experimental, diagnostic, prophylactic, and/or therapeutic purposes, should the subject be virally infected or otherwise in need thereof. Typical subjects include any animal (e.g., mammals such as mice, rats, rabbits, non-human primates, and humans). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

The identification of a particular active agent as having a certain activity is not limiting, unless otherwise indicated, and does not preclude the same agent from having additional activities.

Pharmaceutical compositions of the disclosure comprise interferons (IFNs), methods of treating or preventing viral respiratory infections in subjects thereof using IFNs thereby reducing or minimizing viral respiratory infections, symptoms thereof, inflammation, and virus propagation. In one embodiment, an inhalable pharmaceutical composition may be the inhalable pharmaceutical composition comprises one or more interferons (IFN); one or more inhalable corticosteroids; one or more aminoquinolines such as 4-aminoquinoline, amodiaquine, chloroquine, or hydroxychloroquine; and one or more pharmaceutically acceptable excipients, carriers, and/or diluents. The one or more interferon is, for example, interferon alpha (IFNα) interferon beta (IFNβ), either individually or in combination. The inhalable pharmaceutical composition may optionally further comprise one or more therapeutic agent (e.g., antibiotics, anti-inflammatories, antivirals, anti-parasitics, and mucoactive agents, or other agents that assist or enhance the effectiveness of the composition (e.g., bronchodilators, beta-2 agonists, such as, albuterol, levalbuterol, epinephrine injection, salbutamol, salmeterol, formoterol, vilanterol; anticholinergics, such as ipratropium, tiotropium, aclidinium, glycopyrronium; xanthine derivatives, such as theophylline, aminophylline)).

Another embodiment of the disclosure provides an inhalable pharmaceutical composition comprising at least one interferon (IFN), at least one therapeutic agent, and one or more pharmaceutically acceptable excipients, carriers, or diluents. The at least one interferon of the disclosed pharmaceutical composition may be selected from: a Type I IFN (e.g., IFN-alpha (IFNα), IFN-beta (IFNβ), IFN-epsilon (IFNε), IFN-kappa (IFNκ), IFN-omega (IFNω), IFN-tau (IFNτ), IFN-zeta (ζ)); a Type II IFN (e.g., IFN-gamma (IFNγ)); and a Type III IFN (e.g., IFN-lambda 1 (IFNλ1) (Interleukin-29 [IL-29]), IFN-λ2 (IL29A), IFNλ3 (IL-28B), IFNλ4))). In those embodiments where the at least one interferon is a Type I IFN, the Type I IFN may be selected from: IFNα-1, IFNα2 (e.g., IFNα-2a, IFNα-2b), IFNα4, IFNα5, IFNα6, IFNα7, IFNα8, IFNα10, IFNα13, IFNα14, IFNα16, IFNα17, IFNα21, IFNα-n1, IFNα-n3, IFNβ1 (e.g., IFNβ-1a, IFNβ-1b), and IFNβ3, individually or in combinations of two or more thereof. Another embodiment may provide for the at least one interferon that is IFNγ. In yet other embodiments, the at least one therapeutic agent may be selected from one or more: antibiotics, anti-inflammatories, and antivirals, for example, antibiotics and anti-inflammatories.

Another embodiment may be directed to pharmaceutical compositions of the disclosure comprising the one or more antibiotics selected from: amoxicillin, azithromycin, erythromycin, penicillin, amoxicillin, and cefadroxil, for example, azithromycin, individually or in combinations of two or more thereof. A further embodiment may provide pharmaceutical compositions of the disclosure comprising the one or more anti-inflammatories selected from: chloroquine, 4-aminoquinoline, hydroxychloroquine (e.g., hydroxychloroquine sulfate), ibuprofen, naproxen, celecoxib, oxaprozin, piroxicam, aspirin (acetylsalicylic acid (ASA)), diclofenac, inhibitors of cyclo-oxygenase-1 (COX-1), COX-2, IL-1β, IL-2, IL-6, IL-7, IL-8, IL-10, IL-12, tumor necrosis factor α (TNF-α), granulocyte-colony stimulating factor (G-CSF), interferon-γ-inducible protein (IP10), monocyte chemoattractant protein (MCP1), and macrophage inflammatory protein 1 alpha (MIP1A), individually or in combinations of two or more thereof. In yet another embodiment, pharmaceutical compositions of the disclosure may comprise the one or more antivirals selected from chloroquine; 4-aminoquinoline; hydroxychloroquine (e.g., hydroxychloroquine sulfate); chlorpromazine; loperamide; lopinavir; lycorine; emetine; monensin sodium; mycophenolate mofetil; mycophenolic acid; phenazopyridine; pyrvinium pamoate; OYA1 (OyaGen, Inc.); remdesivir ((2S)-2-{(2R,3S,4R,5R)-[5-(4-Aminopyrrolo[2,1-f][1,2,4]triazin-7-yl)-5-cyano-3,4-dihydroxy-tetrahydro-furan-2-ylmethoxy]phenoxy-(S)-phosphorylamino}propionic acid 2-ethyl-butyl ester); neuraminidase inhibitor (e.g., oseltamivir, peramivir, zanamivir, laninamivir, 2,3-dehydro-2-deoxy-N-acetylneuraminic acid (DANA)); nucleoside analogs (acyclovir, cymevene, ribavirin (1-β-D-ribofuranosyl-1H-1,2,4-triazole-3-carboxamide)); favipiravir (T-705; 6-fluoro-3-hydroxy-2-pyrazinecarboxamide); protease inhibitor (e.g., indinavir, nelfinavir, saquinavir); reverse transcriptase inhibitor (e.g., lamivudine, zidovudine); amantadine; and foscarnet. Yet a further embodiment may provide the at least one IFN is selected from IFNα, IFNβ, and IFNγ; and the at least one therapeutic agent is an antibiotic and an anti-inflammatory, such as for example, azithromycin and hydroxychloroquine, respectively.

The pharmaceutical compositions of the present disclosure may comprise at least one corticosteroid. In some embodiments, the corticosteroid is a glucocorticoid such as an inhaled glucorcorticoid. The corticosteroid may be fluticasone, beclomethasone, budesonide, mometasone, ciclesonide, flunisolide, triamcinolone, prednisone, prednisolone, methylprednisone, dexamethasone or hydrocortisone. The corticosteroid may be an inhalable corticosteroid suitable for administration by inhalation or oral corticosteroids which are corticosteroids suitable for oral administration. Exemplary inhalable corticosteroids are fluticasone, beclomethasone, budesonide, mometasone, ciclesonide, flunisolide, or triamcinolone. The corticosteroid may be present as a pharmaceutically acceptable salt such as fluticasone propionate, fluticasone furoate, beclomethasone dipropionate, mometasone furoate, and triamcinolone acetonide. Representative oral corticosteroids include prednisone, prednisolone, methylprednisone, dexamethasone or hydrocortisone, including their pharmaceutically acceptable salts, solvates, and physical forms.

Another embodiment of the disclosure may be directed to a method of treating or preventing a viral respiratory infection (e.g., coronavirus, SARS-CoV, SARS-CoV2, MERS-CoV) in a subject in need thereof comprising administering a therapeutically effective amount of any one of the disclosed pharmaceutical compositions. In one embodiment, the method of treating or preventing a viral respiratory invention in a subject in need thereof may provide for administering a therapeutically effective amount of any one of the disclosed pharmaceutical compositions, where the pharmaceutical composition is inhalable.

In some embodiments, the method may comprise administrating (e.g., inhalable administration):
(a) an IFN (e.g. inhalable IFN);
(b) aminoquinoline;
(c) optionally a corticosteroid (e.g., inhalable corticosteroid); and
(d) optionally at least one therapeutic agent,
where any or all of the IFN, aminoquinoline, corticosteroid, therapeutic agent may optionally be formulated as an inhalable.
Administration steps (angle)-(d) may be concurrent, sequential, or simultaneous.

Administration of any of the steps (a), (b), (c), and (d) may be performed concurrently, simultaneously, or sequentially. For example, the interferon, aminoquinoline, corticosteroid, and at least one therapeutic agent are each administered sequentially. In some embodiments an interferon, an aminoquinoline, a corticosteroid, and at least one therapeutic agent are each administered simultaneously (e.g., each are present in the same pharmaceutical composition, or each is separately or individually administered). In some embodiments, the method may comprise:
(i) simultaneously administering an inhalable interferon and an inhalable corticosteroid by administration of a pharmaceutical composition comprising
   (1) the inhalable interferon,
   (2) the corticosteroid, and
   (3) one or more pharmaceutically acceptable carriers, excipients, or diluents; and;
   (4) optionally at least one therapeutic agent; and
(ii) administering the inhalable aminoquinoline sequentially with respect to the pharmaceutical composition.

In some embodiments, the method of treating a viral respiratory infection in a subject in need thereof may comprise:
(a) administering an interferon (IFN);
(b) administering an aminoquinoline selected from chloroquine, and hydroxychloroquine; and
(c) administering a corticosteroid.

Administration of any of the steps (a), (b), and (c) may be performed simultaneously or sequentially. For example, the interferon, inhalable corticosteroid, and chloroquine are each administered sequentially. In some embodiments an inhalable interferon, an inhalable corticosteroid, and an inhalable aminoquinoline are each administered simultaneously (e.g., each are present in the same inhalable pharmaceutical composition). In some embodiments, the method may comprise:
(i) simultaneously administering an inhalable interferon and an inhalable corticosteroid by administration of a pharmaceutical composition comprising
   (1) the inhalable interferon,
   (2) the corticosteroid, and
   (3) one or more pharmaceutically acceptable carriers, excipients, or diluents; and
(ii) administering the inhalable aminoquinoline sequentially with respect to the pharmaceutical composition.

In certain implementations, the method may comprise:
(i) simultaneously administering the inhalable interferon and the inhalable aminoquinoline by administration of a pharmaceutical composition comprising:
   (1) the inhalable interferon and,
   (2) the inhalable aminoquinoline, and
   (3) one or more pharmaceutically acceptable carriers, excipients, or diluents; and
(ii) administering the corticosteroid sequentially with respect to the pharmaceutical composition.

In some aspects, the method may comprise:
(i) simultaneously administering the corticosteroid and the inhalable aminoquinoline by administration of a pharmaceutical composition comprising:
   (1) the inhalable interferon and
   (2) the inhalable aminoquinoline, and
   (3) one or more pharmaceutically acceptable carriers, excipients, or diluents; and
(ii) administering the inhalable interferon sequentially with respect to the pharmaceutical composition.

The method may further comprise:

(d) administering at least one therapeutic agent selected from one or more antibiotics, one or more anti-inflammatories, one or more antivirals, one or more anti-parasitic, and one or more mucoactive agents. The one or more antibiotics may be selected from amoxicillin, azithromycin, erythromycin, penicillin, amoxicillin, and cefadroxil. The one or more anti-inflammatories may be selected from ibuprofen, naproxen, celecoxib, oxaprozin, piroxicam, aspirin (acetylsalicylic acid (ASA)), diclofenac, inhibitors of cyclo-oxygenase-1 (COX-1), COX-2, IL-1β, IL-2, IL-6, IL-7, IL-8, IL-10, IL-12, tumor necrosis factor α (TNF-α), granulocyte-colony stimulating factor (G-CSF), interferon-7-inducible protein (IP10), monocyte chemoattractant protein (MCP1), and macrophage inflammatory protein 1 alpha (MIP1A). In some embodiments, the one or more antivirals are selected from chlorpromazine, loperamide, lopinavir, lycorine, emetine, monensin sodium, mycophenolate mofetil, mycophenolic acid, phenazopyridine, pyrvinium pamoate, OYA1 (OyaGen, Inc.), remdesivir ((2S)-2-{(2R,3S,4R,5R)-[5-(4-Aminopyrrolo[2,1-f][1,2,4]triazin-7-yl)-5-cyano-3,4-dihydroxy-tetrahydro-furan-2-ylmethoxy]phenoxy-(S)-phosphorylamino}propionic acid 2-ethyl-butyl ester), neuraminidase inhibitor, nucleoside analogs, favipiravir, protease inhibitor, reverse transcriptase inhibitor, amantadine, and foscarnet. The one or more anti-parasitics may be selected from anti-malarials, anti-babesials, anti-amoebics, anti-giardials, trypanocidals, anti-leishmanials, anti-toxoplasma agents, antipneumocystis agents, anti-trichomoniasis agents, anti-helminthics, anti-cestodals, anti-nematodals, and anti-scabietics, and pediculicides. In some implementations, the one or more mucoactive agents are selected from expectorants, mucolytics, mucokinetics, and mucoregulators. For example, the one or more mucoactive agents may be selected from guaifenesin, potassium iodide, acetylcysteine, sodium citrate, potassium citrate, Tolu balsam, vasaka, ammonium chloride, ambroxol, bromhexine, carbocisteine, erdosteine, mecysteine, and dornase alfa.

In particular embodiments, administration of one or more of the actives may occur via inhalation of the active ingredients. For example, the actives may be administered in a spray, aerosol, liquid, dry powder, particles, or droplets. Inhaled administration may target deposition of one or more of the actives in the lungs. For example, the spray, aerosol, liquid, dry powder, particles, or droplets may have a particle size in a range of from 0.5 μm to 5 μm (e.g., as measured by dynamic light scattering).

In some embodiments, a method of treating COVID-19 in a subject in need thereof is provided which may comprise:
(a) administering an inhalable IFNα or IFNβ;
(b) administering inhalable azithromycin,
(c) administering inhalable hydroxychloroquine; and
(d) administering an inhalable corticosteroid. The inhalable IFNα, inhalable IFNβ, azithromycin, and hydroxychloroquine, may each be independently administered simultaneously, sequentially, or in sequential combinations of any two or more thereof.

Any administration may occur independently with respect to sequential administration. For example, the active ingredients (or pharmaceutical compositions) of the present disclosure such as the interferons, aminoquinolines, corticosteroids, or therapeutic agents may be independently administered for example, one or more times in one day (e.g., 1, 2, 3, 4, 5, 6, 7 times per day). The active may be administered over a period of time of, for example, 1, 2, 3, 4, 5, 6, 7 days, or even longer with a certain frequency, such as hourly, twice daily, daily, biweekly, weekly, or every two weeks. In some embodiments, an indicated active (or combination of actives such as two or more of interferon, aminoquinoline, or corticosteroid) may be administered for 1, 2, 3, 4, 5, 6, 7 days or longer with a specified frequency (e.g., hourly, twice daily, daily, biweekly, weekly, or every two weeks) followed by sequential administration of another active (or combination of actives) for 1, 2, 3, 4, 5, 6, 7 days, or longer with a specified frequency (e.g., hourly, twice daily, daily, biweekly, weekly, or every two weeks). Administration may occur, for example, for 1, 2, 3, 4 weeks, or even longer. One or more dosage forms can be administered, for example, for 1, 2, 3, 4, 5, 6, 7, 8, 9, 10, 11, 12 months, or even longer. In certain embodiments, sequential administration may occur on a per dose basis, such as one or more doses (e.g., two, three, four, five, six, seven eight) of a certain active (or combination of actives) may be administered following one or more doses (e.g., two, three, four, five, six, seven eight) of another active (or combination of actives.

One or more dosage forms can be administered until the patient, subject, mammal, mammal in need thereof, human, or human in need thereof, does not require treatment, prophylaxis, or amelioration of any disease or condition such as, for example, viral respiratory infections. In some embodiments, the dosage form may be co-administered (i.e., substantially simultaneously or simultaneously) or sequentially administered with other pharmaceutical compositions comprising one or more therapeutic agents until the patient, subject, mammal, mammal in need thereof, human, or human in need thereof, does not require further treatment, prophylaxis, or amelioration of any disease or condition, such as, for example, viral respiratory infections. In various embodiments, the pharmaceutical compositions comprising one or more IFNs and/or one or more therapeutic agents (e.g., antibiotics, anti-inflammatories, antivirals) administered simultaneously or substantially simultaneously or sequentially in a specified period greater than 10 minutes (e.g., greater than 15 minutes) or in a period of 1-hour, 2-hour, 4-hour, 6-hour, 12-hour, 24-hour, 36-hour, 72-hour, or one or more times a week, or the like.

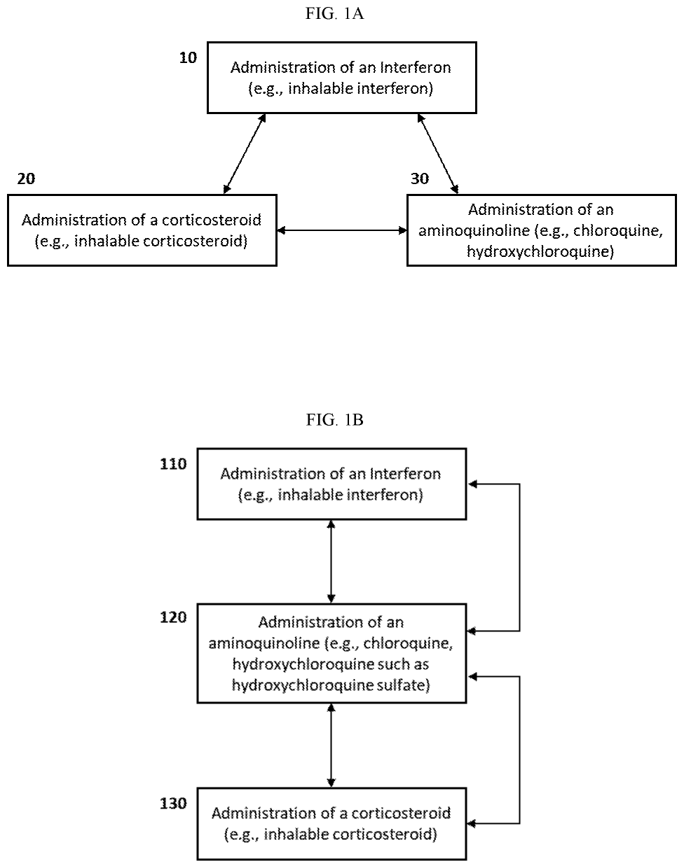

Referring now to FIG. 1A, a flowchart of an administration regimen of several actives of the present disclosure is illustrated. A subject in need thereof may be administered an interferon at step 10. Subsequently, or simultaneously to step 10, a subject in need thereof may be administered a corticosteroid at step 20 or be administered an aminoquinoline at step 30. Similar embodiments are depicted when the corticosteroid is administered first at step 20 or when the aminoquinoline is administered first at step 30. In certain embodiments, the first administration is a combination of the indicated steps such as a combination of step 10 and step 20, a combination of step 10 and step 30, a combination of step 20 and step 30. In certain embodiments, each step occurs simultaneously (e.g., by administration of an inhalable pharmaceutical composition comprising the interferon (e.g., inhalable interferon), the corticosteroid (e.g., inhalable corticosteroid), and aminoquinoline (e.g., inhalable aminoquinoline). In certain embodiments, the subject in need thereof may first be administered the aminoquinoline at step 20, the corticosteroid at step 30, either alone, in combination, or in combination with administration of the interferon at step 10. Sequential and simultaneous permutations of the administration steps are FIG. 1B illustrates an exemplary administration regimen wherein the interferon is administered initially alone (step 110) or in combination with one or more of the aminoquinoline (step 120) and the corticosteroid (step 130). The present disclosure is not restricted by the order or frequency of steps unless expressly stated. An embodiment involves sequential administration of one or more administering steps and another embodiment involves simultaneous administration one or more administering steps.

A further embodiment provides for a method of treating or preventing a viral respiratory infection in a subject in need thereof comprising administering an inhalable pharmaceutical composition comprising a therapeutically effective amount of at least one interferon (IFN) and one or more pharmaceutically acceptable excipients, carriers, and/or diluents. Other embodiments may provide for the administration of the at least one IFN elected from: a Type I IFN (e.g., IFN-alpha (IFNα), IFN-beta (IFNβ), IFN-epsilon (IFNε), IFN-kappa (IFNκ), IFN-omega (IFNω), IFN-tau (IFNτ), IFN-zeta (ζ)); a Type II IFN (e.g., IFN-gamma (IFNγ)); and a Type III IFN (e.g., IFN-lambda 1 (IFNλ1) (Interleukin-29 [IL-29]), IFN-λ2 (IL29A), IFNλ3 (IL-28B), IFNλ4). In yet a further embodiment, the method may comprise the at least one interferon may be selected from: IFN α, IFNβ, and IFNγ, and any subtypes thereof. The methods of the disclosure may further comprise administering a therapeutically effective amount of at least one therapeutic agent, where the at least one interferon and the at least one therapeutic agent may be administered simultaneously, substantially simultaneously, or sequentially, by the same or different routes of administration, where the at least one therapeutic agent is selected from one or more: antibiotics, anti-inflammatories, and antivirals. One embodiment may be directed to a method of the disclosure where the at least one therapeutic agent is one or more antibiotics and one or more anti-inflammatories. The antibiotics may be selected from: amoxicillin, azithromycin, erythromycin, penicillin, amoxicillin, and cefadroxil; the anti-inflammatories may be selected from: chloroquine, 4-aminoquinoline, hydroxychloroquine (e.g., hydroxychloroquine sulfate), ibuprofen, naproxen, celecoxib, oxaprozin, piroxicam, aspirin (acetylsalicylic acid (ASA)), diclofenac, inhibitors of cyclo-oxygenase-1 (COX-1), COX-2, IL-1β, IL-2, IL-6, IL-7, IL-8, IL-10, IL-12, tumor necrosis factor α (TNF-α), granulocyte-colony stimulating factor (G-CSF), interferon-γ-inducible protein (IP10), monocyte chemoattractant protein (MCP1), and macrophage inflammatory protein 1 alpha (MIP1A); and the antivirals may be selected from: chloroquine, 4-aminoquinoline; hydroxychloroquine (e.g., hydroxychloroquine sulfate); chlorpromazine; loperamide; lopinavir; lycorine; emetine; monensin sodium; mycophenolate mofetil; mycophenolic acid; phenazopyridine; pyrvinium pamoate; OYA1 (OyaGen, Inc.); remdesivir ((2S)-2-{(2R,3S,4R,5R)-[5-(4-Aminopyrrolo[2,1-f][1,2,4]triazin-7-yl)-5-cyano-3,4-dihydroxy-tetrahydro-furan-2-ylmethoxy]phenoxy-(S)-phosphorylamino}propionic acid 2-ethyl-butyl ester); neuraminidase inhibitor (e.g., oseltamivir, peramivir, zanamivir, laninamivir, 2,3-dehydro-2-deoxy-N-acetylneuraminic acid (DANA)); nucleoside analogs (acyclovir, cymevene, ribavirin (1-β-D-ribofuranosyl-1H-1,2,4-triazole-3-carboxamide)); favipiravir (T-705; 6-fluoro-3-hydroxy-2-pyrazinecarboxamide); protease inhibitor (e.g., indinavir, nelfinavir, saquinavir); reverse transcriptase inhibitor (e.g., lamivudine, zidovudine); amantadine; and foscarnet.

Another aspect of the disclosure provides for the administration of the IFN in a therapeutically effective amount in an amount of 1 picogram (pg)/milliliter (mL)-100 micrograms (μg)/mL (e.g., 100 pg/mL-50 μg/mL, 1 nanogram (ng)/mL-1 μg/mL, 10 ng/mL-100 ng/mL) and the therapeutic agent in a therapeutically effective amount in an amount of 1 μg/kg/day-1000 mg/kg/day (e.g., 10 μg/kg/day-750 mg/kg/day, 100 μg/kg/day-500 mg/kg/day, 500 μg/kg/day-100 mg/kg/day).

The pharmaceutical composition may be in unit dose form (e.g. spray, liquid, aerosol, dry powder, gas, atomizable particles or droplets). In certain embodiments the unit dose for comprises IFN in a therapeutically effective amount in an amount of 1 picogram (pg)/milliliter (mL)-100 micrograms (μg)/mL (e.g., 100 pg/mL-50 μg/mL, 1 nanogram (ng)/mL-1 μg/mL, 10 ng/mL-100 ng/mL) or alternatively, IFN may be 0.5 million international units (IU)-10 million IU (e.g., 1 million IU-8 million IU, 2 million IU-6 million IU) and the therapeutic agent in a therapeutically effective amount in an amount of 1 μg/kg/day-1000 mg/kg/day (e.g., 10 μg/kg/day-750 mg/kg/day, 100 μg/kg/day-500 mg/kg/day, 500 μg/kg/day-100 mg/kg/day). The dosages may be administered on a once or more weekly basis, or on a once or more daily basis, or the like as determined by a medical practitioner.

The pharmaceutical compositions of the present disclosure may also be in the form of an oral or nasal spray. The oral or nasal spray may be formulated such that each spray administers, for example, less than 100 micrograms/mL, less than 50 micrograms/mL, less than 1 microgram/mL, less than 100 nanograms/mL, or less than 100 picograms/mL of IFN. The oral, nasal, or inhaled spray may further comprise a therapeutic agent sprayed in an amount of less than 1000 mg/kg/day, less than 500 mg/kg/day, less than 50 mg/kg/day, less than 1 mg/kg/day, less than 100 μg/kg/day, less than 10 μg/kg/day, less than 1 μg/kg/day. The spray, liquid, aerosol, dry powder, gas, atomizable particles or droplets may be in a volume ranging from 1 milliliter to 50 milliliters and contain particles, comprising the pharmaceutical composition of the disclosure, in a particle size range of 0.5 micron-5 microns (e.g., as measured by dynamic light scattering), as these are sizes used for aerosols that are intended to be targeted into the lung or lower respiratory tract, which is useful for respiratory disease, conditions, or the like, including but not limited to viral respiratory infections. Another embodiment may utilize particle sizes in a range from greater than 5 microns (e.g., 10-100 microns, 10-50 microns, 10-30 microns) for optimal delivery to the nasal region or upper respiratory tract.

The active ingredients of the present disclosure such as the interferons, aminoquinolines, corticosteroids, or therapeutic agents may be administered to a subject in a “therapeutically effective amount” that is sufficient to demonstrate a benefit to the subject either alone or in combination with one another. The actual amount administered, and rate and time-course of administration, will depend on the nature and severity of the viral respiratory infection being treated. Prescriptions of treatment, e.g. decisions on dosage, frequency, etc, is ultimately within the responsibility and at the discretion of general practitioners and other medical doctors, and generally accounts for the disorder to be treated, the condition of the individual subject or patient in need thereof, the site of delivery, the method of administration and other factors known to practitioners.

The optimal dose may be determined by physicians or other medical practitioners based on a several parameters including, but not limited to, age, sex, weight, severity of the condition being treated, the active ingredient being administered, and the route of administration.

Another embodiment of the disclosure provides inhalable pharmaceutical compositions formulated as: an oral spray, a nasal spray, an aerosol, a liquid, a dry powder, a gas, or atomizable particles or droplets. The administering step of the method disclosed here may utilize a nebulizer, inhaler (e.g., metered-dose inhalers, dry-powder inhalers), gas masks (SootherMask™; InspiraMask™; InspiRx, Inc.), or the like.

Other embodiments of the methods of the disclosure may treat, prevent, and/or reduce viral respiratory infections caused by viruses selected from: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV). In yet further embodiments, the viral respiratory infection may be selected from: a cold, bronchiolitis, croup, pneumonia, coronavirus disease 2019 (e.g., COVID-19), severe acute respiratory syndrome (SARS), middle East respiratory syndrome (MERS).

One embodiment of the disclosure provides a method of treating, preventing, or reducing COVID-19 in a subject in need thereof comprising administering an inhalable IFNα and/or inhalable IFNβ in a therapeutically effective amount; and administering azithromycin and/or hydroxychloroquine. An additional embodiment may be directed to the method of the disclosure where administrating the inhalable IFNα and/or inhalable IFNβ and administering azithromycin and/or hydroxychloroquine occurs simultaneously or sequentially.

In some embodiments, the total IFN content of the composition may be more than 70%, more than 80%, more than 90%, more than 95%, or more than 99% IFN by weight of the total IFN content.

Additional ingredients may be present in the pharmaceutical compositions of the disclosure, including but not limited to ingredients that aid in the administration and effectiveness of the pharmaceutical composition in treating the viral respiratory infection (e.g., coronavirus, COVID-19). Pharmaceutical compositions according to the disclosure, and for use in accordance with the present invention may comprise, in addition to the active ingredient (i.e. one or more interferons or therapeutic agents), a pharmaceutically acceptable excipient, carrier, buffer stabilizer or other materials well known to those skilled in the art may be included. These materials should be non-toxic and should not interfere with the efficacy of the active ingredient or therapeutic agents of the disclosure. The exact nature of the carrier or other material depends on the selected route of administration, which may be, for example, oral, intravenous, or intranasal.

The pharmaceutical composition of the disclosure may be a liquid, for example, a physiologic salt solution containing non-phosphate buffer at pH 6.4 to 7.6, or a lyophilized powder.

Other embodiments may be directed to a pharmaceutical composition having a weight ratio of an interferon to a different interferon, or an interferon (or combination of interferons) to a therapeutic agent (including a combination of therapeutic agents), from 10:1 to 1:10 (e.g., 9:1 to 1:9, 7:1 to 1:7, 2:1 to 1:2, 3:2 to 2:3).

The pharmaceutical compositions according to the disclosure may be in the form of a spray (e.g., an oral or nasal spray), a dry powder, an aerosol, a liquid, a gas, or atomizable particles or droplets. These formulations may be used in combination with nebulizers, inhalers (e.g., metered-dose inhalers, dry-powder inhalers), gas masks (SootherMask™; InspiraMask™; InspiRx, Inc.), or the like. The desired formulations comprising the active ingredient and/or therapeutic agent may comprise vehicles, carriers, or the like that are particularly selected to provide enhance contact time between the pharmaceutical composition in the subject, for example in the nasal or respiratory tract for a sufficient time period to enact its benefits. This time period may be at least 1 minute or greater (e.g., 5 minutes, 10 minutes, 15 minutes, 20 minutes, 25 minutes, 30 minutes, 1 hour, 2 hours, 4 hours, 6 hours, 12 hours, 24 hours) following application. In some embodiments, the composition for inhalation comprises one or more IFNs and/or one or more therapeutic agents dispersed in a liquid carrier comprising from 1-99% (v/v) water or other pharmaceutically and therapeutically acceptable solvent (e.g., 10-90% (v/v) water; 20-60% (v/v) water, from 30-40% (v/v) water).

The compositions may be administered by any suitable route, including orally, topically, nasally, and combinations thereof. In an embodiment, the composition is administered to nasal membranes. In an embodiment, the composition is administered to oral membranes. In an embodiment, the composition is administered using a device selected from the group consisting of an atomizer, an inhaler, a nebulizer, a gas mask, a spray bottle, and a spray pump. The composition may also include a propellant or may be free of propellants.

The compounds and pharmaceutical compositions can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

The pharmaceutical compositions may contain one or more additional components, for example, sweetening agents such as sucrose, fructose, lactose, aspartame or saccharin; flavoring agents such as peppermint, oil of wintergreen, or cherry; coloring agents; pH adjusting component, humectants, and preserving agents, to provide a pharmaceutically palatable preparation. Typical sweetening agents (sweeteners) useful in the composition include those that are both natural and artificial sweeteners. Sweetening agent used may be selected from a wide range of materials including water-soluble sweetening agents, water-soluble artificial sweetening agents, water-soluble sweetening agents derived from naturally occurring water-soluble sweetening agents, dipeptide based sweetening agents, and protein based sweetening agents, including mixtures thereof. Representative examples of moisturizing or humectant agents that are usable in the present invention include, without limitation, acetamide monoethanolamine urazole, aloe vera in any of its variety of forms (e.g., aloe vera gel, aloe vera extract, aloe vera concentrate), allantoin, guanidine, glycolic acid and glycolate salts (e.g., ammonium salt and quaternary alkyl ammonium salt), hyaluronic acid, lactamide monoethanolamine, polyethylene glycols, polyhydroxy alcohols (e.g., sorbitol, glycerol, hexanetriol, propylene glycol, butylene glycol, hexylene glycol and the like), sugars and starches, sugar and starch derivatives (e.g., alkoxylated glucose), and any combination thereof. Suitable flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing. Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing. In addition to the flavoring and sweetening agents, the compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol. In some embodiments, the pharmaceutical composition may comprise one or more binders, such as, for example, carboxymethylcellulose, alginates, gelatin, polyvinyl pyrrolidone, sucrose and/or acacia.

The pharmaceutical composition may comprise one or more natural extracts and concentrates. Suitable whole leaf aloe vera concentrate may, for example, act as a carrying agent. The whole leaf aloe vera concentrate is present in an amount less than 10% (w/v) of the pharmaceutical composition, for example, from 2% (w/v) to 4% (w/v) or 0.1% (w/v) to 3% (w/v) or from 0.1% (w/v) to 2% (w/v) of the pain relieving composition. Although some studies may show that aloe extracts may confer anti-inflammatory properties, in some embodiments, the aloe is present in an amount less than is efficacious for such activity. Accordingly, the aloe may be considered part of the IFN content or not part of the IFN content, dependent on the concentration and dosage administered. In most embodiments, aloe extract is not considered part of the IFN content. In some embodiments, the pharmaceutical composition comprises less than 10% (w/v) aloe.

In some embodiments, the pharmaceutical composition of the disclosure, such as for oral or nasal sprays, may be a liquid or solution including an aqueous solution where the IFN(s) and/or therapeutic agent(s) may be buffered using saline, acetate, phosphate, citrate, acetate or other buffering agents, which may be at any physiologically acceptable pH, generally from about pH 4 to about pH 7. Combinations of buffering agents may also be employed, such as phosphate buffered saline, a saline and acetate buffer, and the like. In the case of saline, a 0.9% saline solution may be employed. In the case of acetate, phosphate, citrate, acetate and the like, a 50 mM solution may be employed. In addition to buffering agents, suitable preservatives may be employed, to prevent or limit bacteria and other microbial growth.

In preferred embodiments, the pharmaceutical composition is administered orally, and more particularly, as an oral spray. A sweetener and flavor enhancers may also be included in the oral spray composition. Sweeteners may include fructose, dextrose, sucrose or the like. Non-artificial sweeteners work best with a preferred embodiment including fructose in an amount of from 8 to 15 weight percent of the oral spray composition, and preferably at 10 weight percent of the oral composition. One certain embodiment of the oral spray composition includes a flavor enhancer, such as peppermint, for example, in an amount of 0.5 to 2.0% (w/w) of the oral spray composition, including 1% (w/w) of the oral composition.

In accordance with another aspect of the present disclosure, a preservative may be added to the pharmaceutical composition to facilitate stability of the various ingredients. Any suitable preservative may be used in accordance with the present disclosure such as, for example, benzalkonium chloride, benzyl alcohol, and disodium EDTA. The preservative may include a 50% solution of benzalkonium chloride mixed into the pharmaceutical composition of the disclosure at a concentration of 0.01% by weight to 1% by weight, for example 0.5% by weight.

In certain embodiments, the pharmaceutical composition of the disclosure may be formulated with at least one IFN (e.g., alpha, beta, gamma) to achieve a therapeutic dose of IFN for treating subjects having a viral respiratory infection. In some embodiments, the pharmaceutical composition may be in the form of an oral, nasal, or respiratory spray composition, such that the composition may be delivered to the upper and lower respiratory tract. The spray composition may be used to deliver from 100 μl to 50 mL (e.g., from 500 μl-10 mL) of the active ingredient per activation from an appropriate apparatus, such as but not limited to, an inhaler, nebulizer, aerosol spray, gas mask, and the like.

In a further embodiment, the present disclosure relates to system comprising a stable pre-metered dose of a pharmaceutical composition of the instant disclosure, where the pre-metered dose may be in a container for nasal or oral or inhaled administration. The system may further comprise a package insert containing instructions regarding the use of the container for releasing or administering the pharmaceutical composition.

In one embodiment, the container is part of a sprayer, inhaler, or nebulizer may have an actuator. When the actuator is actuated, the composition is delivered in the form of a spray or mist. In a further embodiment, the pharmaceutical composition is contained in a sprayer, inhaler, or nebulizer or gas mask, that delivers a spray or mist comprising the pharmaceutical composition to a human nose in an amount and means sufficient to deliver a therapeutically effective amount. In the instant disclosure, the pharmaceutical composition when inhaled or delivered as a nasal and/or oral spray or mist using an inhaler, sprayer, nebulizer, or gas mask, results in a spray pattern and droplet size sufficient to maximize the delivered amount. The spray patterns and droplet size may be determined by any of a number of techniques, including but not limited to an axisymmetric drop shape analysis (ADSA) with Nasal Spray Products Universal Actuator (NSP UA) set up (Innova System) and a Malvern Spraytec with NSPUA set up (Innova System) for determining the spray droplet size distribution. Typical and commonly used protocols may be used for determining droplet size distribution of the spray.

In some embodiments, the aqueous suspension is provided in the form of an oral spray, nasal spray, inhalation spray or mist, wherein the suspension is administered in a single unit-dose container or multi-dose container that is pre-metered or pre-determined. Suitable single unit-dose containers or multi-dose containers include, but are not limited to, glass, aluminum, polypropylene, or high-density polyethylene, for example, high density polyethylene containers produced using a blow-fill-seal manufacturing technique.

The composition of the present disclosure may be delivered to the upper and/or lower respiratory tract through the mouth and/or nose by way of a fine spray mist. The inhalable pharmaceutical composition may also be administered in microspheres, liposomes, other microparticulate delivery systems as delivered to particular tissues of the subject, for example, the upper and/or lower respiratory tracts. Suitable examples of sustained release carriers may include semipermeable polymer matrices in the form of microcapsules. The method includes the steps of obtaining an inhalable pharmaceutical composition in accordance with the instant disclosure for delivery into the upper (e.g., nasal cavity, pharynx, larynx) and/or lower respiratory tract (e.g., trachea, primary bronchi, lungs). The method further includes the step of administering the inhalable pharmaceutical composition using a spray applicator, inhaler, metered dose inhaler (MDI), nebulizer, gas mask, or the like. Practitioners will appreciate that any suitable applicator may be used. For example, the applicator may be configured to hold from 10-100 metered doses of the composition, wherein the metered dose is from 0.1 mL to 10 mL (e.g., from 0.25 mL to 5 mL, 0.5 mL to 1 mL).

Inhalation delivery systems are commonly known and used. For example, MDIs may comprise the pharmaceutical composition of the disclosure in a pre-metered dosage amount. In some embodiments, the MDI may also contain a propellant or excipient(s). The canister is may be filled with a suspension of an active agent, such as an oral, nasal, or inhalable spray composition as described herein, and a propellant, such as one or more hydrofluoroalkanes [e.g. 1,1,1,2-tetrafluoroethane (HFA-134a) and 1,1,1,2,3,3,3-heptafluoropropane (HFA-227)], chlorofluorocarbons, and alcohols such as ethanol, isopropanol, butanol, propanol or mixtures thereof. However, in one embodiment, the pharmaceutical composition is free of propellants. When the actuator is depressed a metered dose of the suspension comprising the active ingredients (e.g., IFN(s) and optionally one or more therapeutic agents) may be aerosolized for inhalation. Particles comprising the active agent may be propelled towards the mouthpiece where they may then be inhaled by a subject in need thereof. In some embodiments, the particle size is sufficient to deliver the pharmaceutical composition containing at least one interferon (IFN) and one or more pharmaceutically acceptable excipients, carriers, and/or diluents, and optionally at least one therapeutic agent (e.g., antibiotics, anti-inflammatories, antivirals, anti-parasitics, mucoactive agents) to the upper and/or lower respiratory tract. The therapeutic agent may be delivered simultaneously, substantially simultaneously, or sequentially and/or separately from the pharmaceutical composition containing at least one interferon (IFN) and one or more pharmaceutically acceptable excipients, carriers, and/or diluents. Therapeutic agents of the disclosure may include, but are not limited to antibiotics, anti-inflammatories, antivirals, anti-parasitics, and mucoactive agents. In some embodiments, the inhalable composition comprising at least one interferon (IFN) and one or more pharmaceutically acceptable excipients, carriers, and/or diluents, where the at least one IFN is IFNα and IFNβ, may optionally also contain or separately be administered to a subject in need thereof. Further embodiments may be directed to the inhalable composition or methods of using the inhalable composition further comprising or further administering at least one therapeutic agent (e.g., antibiotics, anti-inflammatories, antivirals, anti-parasitics, mucoactive agents, or other enhancing agents), where the antibiotic may be azithromycin, anti-inflammatory, anti-viral, or anti-parasitic may be hydroxychloroquine, and in some embodiments mucoactive agents such as expectorants or mucolytics, and other enhancing agents, such as bronchodilators (e.g., beta-2 agonists, such as, albuterol, levalbuterol, epinephrine injection, salbutamol, salmeterol, formoterol, vilanterol; anticholinergics, such as ipratropium, tiotropium, aclidinium, glycopyrronium; xanthine derivatives, such as theophylline, aminophylline). Mucoactive agents may be used in a separate pharmaceutical composition or regimen to facilitate the breakdown of excessive mucus production resulting from the viral infection. Non-limiting exemplary mucoactive agents include: expectorants, mucolytics, mucokinetics, mucoregulators, or more particularly, guaifenesin, potassium iodide, acetylcysteine, sodium citrate, potassium citrate, Tolu balsam, vasaka, ammonium chloride, ambroxol, bromhexine, carbocisteine, erdosteine, mecysteine, and dornase alfa.

The inhalable pharmaceutical composition comprising the at least one interferon (IFN) and one or more pharmaceutically acceptable excipients, carriers, and/or diluents, and optionally at least one therapeutic agent (e.g., antibiotics, anti-inflammatories, antivirals, anti-parasitics, mucoactive agents, enhancing agents, such as bronchodilators), may be delivered to a subject in any suitable and therapeutically effective dosage. In accordance with one embodiment of the disclosure, the oral, nasal, or inhalable spray applicator, inhaler, nebulizer, or gas mask, may be configured to supply a unit dose of from 0.1 mL to 10 mL (e.g., from 0.25 mL to 5 mL, 0.5 mL to 1 mL) of the inhalable composition to the subject each time a pump associated with the spray applicator is activated (e.g., 0.5 mL/spray). In certain embodiments, the inhalable composition may be delivered by pumping or actuating the device two times to emit 2 sprays in the mouth or nose within 1 minute to 30 minutes.

Administration of the inhalable pharmaceutical composition of the disclosure may vary and be adjusted in accordance with commonly used techniques, formulations, and delivery methods.

### SPECIFIC EMBODIMENTS

Non-limiting specific embodiments are described below each of which is considered to be within the present disclosure.

Specific embodiment 1. A pharmaceutical composition comprising at least one interferon (IFN) and one or more pharmaceutically acceptable excipients, carriers, and/or diluents, and optionally at least one therapeutic agent (e.g., antibiotics, anti-inflammatories, antivirals, anti-parasitics, corticosteroids, mucoactive agents).

Specific embodiment 2. A pharmaceutical composition comprising:
(a) at least one interferon (IFN);
(b) an aminoquinoline selected from chloroquine, hydroxychloroquine, individually or in combination;
(c) a corticosteroid; and
(d) one or more pharmaceutically acceptable excipients, carriers, and/or diluents.

Specific embodiment 3. The pharmaceutical composition according to specific embodiment 1 or 2, wherein the at least one interferon is selected from: a Type I IFN (e.g., IFN-alpha (IFNα), IFN-beta (IFNβ), IFN-epsilon (IFNε), IFN-kappa (IFNκ), IFN-omega (IFNω), IFN-tau (IFNτ), IFN-zeta (ζ)); a Type II IFN (e.g., IFN-gamma (IFNγ)); and a Type III IFN (e.g., IFN-lambda 1 (IFNλ1) (Interleukin-29 [IL-29]), IFN-λ2 (IL29A), IFNλ3 (IL-28B), IFNλ4).

Specific embodiment 4. The pharmaceutical composition according to any one of specific embodiments 1-3, wherein the at least one interferon is a Type I IFN.

Specific embodiment 5. The pharmaceutical composition according to any one of specific embodiments 1-4, wherein the at least one interferon is IFNα and/or IFNβ.

Specific embodiment 6. The pharmaceutical composition according to any one of specific embodiments 3-4, wherein the Type I IFN is selected from: IFNα-1, IFNα2 (e.g., IFNα-2a, IFNα-2b), IFNα4, IFNα5, IFNα6, IFNα7, IFNα8, IFNα10, IFNα13, IFNα14, IFNα16, IFNα17, IFNα21, IFNα-n1, IFNα-n3, IFNβ1 (e.g., IFNβ-1a, IFNβ-1b), and IFNβ3.

Specific embodiment 7. The pharmaceutical composition according to any one of specific embodiments 1-3, wherein the at least one interferon is IFNγ.

Specific embodiment 8. The pharmaceutical composition according to any one of specific embodiments 1-7, wherein the at least one therapeutic agent is selected from one or more: antibiotics, anti-inflammatories, antivirals, anti-parasitics, and mucoactive agents.

Specific embodiment 9. The pharmaceutical composition according to any one of specific embodiments 1-8, wherein the at least one therapeutic agent is one or more antibiotics and one or more anti-inflammatories.

Specific embodiment 10. The pharmaceutical composition according to any one of specific embodiments 8-9, wherein the one or more antibiotics is selected from: azithromycin, erythromycin, roxithromycin, clarithromycin, penicillin, amoxicillin, and cefadroxil.

Specific embodiment 11. The pharmaceutical composition according to any one of specific embodiments 8-9, wherein the one or more anti-inflammatories is selected from: chloroquine, 4-aminoquinoline, hydroxychloroquine (e.g., hydroxychloroquine sulfate), ibuprofen, naproxen, celecoxib, oxaprozin, piroxicam, aspirin (acetylsalicylic acid (ASA)), diclofenac, inhibitors of cyclo-oxygenase-1 (COX-1), COX-2, IL-1β, IL-2, IL-6, IL-7, IL-8, IL-10, IL-12, tumor necrosis factor α (TNF-α), granulocyte-colony stimulating factor (G-CSF), interferon-γ-inducible protein (IP10), monocyte chemoattractant protein (MCP1), and macrophage inflammatory protein 1 alpha (MIP1A).

Specific embodiment 12. The pharmaceutical composition according to specific embodiment 8, wherein the one or more antivirals is selected from: chloroquine, 4-aminoquinoline; amodiaquine; hydroxychloroquine (e.g., hydroxychloroquine sulfate); chlorpromazine; loperamide; lopinavir; lycorine; emetine; monensin sodium; mycophenolate mofetil; mycophenolic acid; phenazopyridine; pyrvinium pamoate; OYA1 (OyaGen, Inc.); remdesivir ((2S)-2-{(2R,3S,4R,5R)-[5-(4-Aminopyrrolo[2,1-f][1,2,4]triazin-7-yl)-5-cyano-3,4-dihydroxy-tetrahydro-furan-2-ylmethoxy]phenoxy-(S)-phosphorylamino}propionic acid 2-ethyl-butyl ester); neuraminidase inhibitor (e.g., oseltamivir, peramivir, zanamivir, laninamivir, 2,3-dehydro-2-deoxy-N-acetylneuraminic acid (DANA)); nucleoside analogs (acyclovir, cymevene, ribavirin (1-β-D-ribofuranosyl-1H-1,2,4-triazole-3-carboxamide)); favipiravir (T-705; 6-fluoro-3-hydroxy-2-pyrazinecarboxamide); protease inhibitor (e.g., indinavir, nelfinavir, saquinavir); reverse transcriptase inhibitor (e.g., lamivudine, zidovudine); amantadine; and foscarnet.

Specific embodiment 13. The pharmaceutical composition according to specific embodiment 8, wherein the one or more anti-parasitic is selected from: anti-malarials (e.g., chloroquine, hydroxychloroquine, amodiaquine and artesunate, atovaquone-proguanil, artemether-lumefantrine, quinine, parenteral quinine), anti-babesials (e.g., atovaquone, clindamycin-quinine), anti-amoebics (iodoquinol, paromomycin sulfate, diloxanide furoate, metronidazole, tinidazole, emetine), anti-giardials (e.g., metronidazole, tinidazole, furazolidone, albendazole), trypanocidals (e.g., nifurtimox, benznidazole, pentamidine, eflornithine, suramin, melarsoprol), anti-leishmanials (e.g., sodium stibogluconate, meglumine antimoniate, pentamidine, amphotericin B, paromomycin), anti-toxoplasma agents (e.g., sulfonamides: sulfadizine, sulfamethazine, sulfamerazine; and pyrimethamine), antipneumocystis agents (trimethoprim-sulfamethoxazole, pentamidine, trimethoprim-dapsone, clindamycin-primaquine), anti-trichomoniasis agents (e.g., metronidazole), anti-helminthics (e.g., vermicides, vermifuges, praziquantel, albendazole), anti-cestodals (praziquantel, niclosamide, albendazole), anti-nematodals (e.g., praziquantel, metrifonate, oxamniquine, bithionol, albendazole, diethylcarbamazine, ivermectin), and anti-scabietics (e.g., lindane, permethrin, benzyl benzoate, ivermectin, permethrin and oral ivermectin, topical ivermectin, synergized pyrethrins), and pediculicides (e.g., permethrin, pyrethrins, malathion, ivermectin)).

Specific embodiment 14. The pharmaceutical composition according to specific embodiment 8, wherein the one or more mucoactive agents is selected from: expectorants, mucolytics, mucokinetics, mucoregulators.

Specific embodiment 15. The pharmaceutical composition according to specific embodiment 8, wherein the one or more mucoactive agents is selected from: guaifenesin, potassium iodide, acetylcysteine, sodium citrate, potassium citrate, Tolu balsam, vasaka, ammonium chloride, ambroxol, bromhexine, carbocisteine, erdosteine, mecysteine, and dornase alfa.

Specific embodiment 16. The pharmaceutical composition according to any one of specific embodiments 1-10, wherein the at least one IFN is selected from IFNα, IFNβ, and IFNγ, either alone or together; and the at least one therapeutic antibiotic is an antibiotic and an anti-inflammatory.

Specific embodiment 17. The pharmaceutical composition according to specific embodiment 16, wherein the antibiotic is azithromycin and the anti-inflammatory is hydroxychloroquine.

Specific embodiment 18. The pharmaceutical composition according to any one of specific embodiments 1-17, wherein the corticosteroid is selected from fluticasone, beclomethasone, budesonide, mometasone, ciclesonide, flunisolide, and triamcinolone.

Specific embodiment 19. The pharmaceutical composition according to any one of specific embodiments 1-18, wherein the pharmaceutical composition is inhalable.

Specific embodiment 20. A method of treating a viral respiratory infection in a subject in need thereof comprising administering a therapeutically effective amount of the pharmaceutical composition according to any one of specific embodiments 1-19.

Specific embodiment 21. A method of treating a viral respiratory infection in a subject in need thereof comprising administering an inhalable pharmaceutical composition comprising a therapeutically effective amount of at least one interferon (IFN) and one or more pharmaceutically acceptable excipients, carriers, and/or diluents, and optionally administering at least one therapeutic agent (e.g., antibiotics, anti-inflammatories, antivirals, anti-parasitics, mucoactive agents).

Specific embodiment 22. A method of treating a viral respiratory infection in a subject in need thereof comprising:
(a) administering an inhalable interferon (IFN);
(b) administering an inhalable aminoquinoline selected from chloroquine, and hydroxychloroquine;
(c) administering an inhalable corticosteroid; and
(d) optionally at least one therapeutic agent.

Specific embodiment 23. The method according to Specific embodiment 22, wherein the inhalable interferon, inhalable corticosteroid, and inhalable aminoquinoline are each administered sequentially.

Specific embodiment 24. The method according to Specific embodiment 22, wherein the inhalable interferon, inhalable corticosteroid, and inhalable aminoquinoline are each administered simultaneously.

Specific embodiment 25. The method according to Specific embodiment 22, wherein
(i) administration of the inhalable interferon and the inhalable aminoquinoline is simultaneous by administration of a pharmaceutical composition comprising:
   (1) the inhalable interferon and,
   (2) the inhalable aminoquinoline, and
   (3) one or more pharmaceutically acceptable carriers, excipients, or diluents; and
(ii) the corticosteroid is administered sequentially with respect to the pharmaceutical composition.

Specific embodiment 26. The method according to Specific embodiment 22, wherein
(i) administration of the inhalable corticosteroid and the inhalable aminoquinoline is simultaneous by administration of a pharmaceutical composition comprising:
   (1) the inhalable corticosteroid and
   (2) the inhalable aminoquinoline, and
   (3) one or more pharmaceutically acceptable carriers, excipients, or diluents;
(ii) the inhalable interferon is administered sequentially with respect to the pharmaceutical composition.

Specific embodiment 27. The method according to any one of specific embodiments 20-26, wherein the at least one IFN is selected from: a Type I IFN (e.g., IFN-alpha (IFNα), IFN-beta (IFNβ), IFN-epsilon (IFNε), IFN-kappa (IFNκ), IFN-omega (IFNω), IFN-tau (IFNτ), IFN-zeta (ζ)); a Type II IFN (e.g., IFN-gamma (IFNγ)); and a Type III IFN (e.g., IFN-lambda 1 (IFNλ1) (Interleukin-29 [IL-29]), IFN-λ2 (IL29A), IFNλ3 (IL-28B), IFNλ4).

Specific embodiment 28. The method according to any one of specific embodiments 20-27, wherein the at least one interferon is a Type I IFN.

Specific embodiment 29. The method according to any one of specific embodiments 20-28, wherein the at least one interferon is IFNα and/or IFNβ.

Specific embodiment 30. The method according to any one of specific embodiments 20-29, wherein the at least one interferon and the at least one therapeutic agent are administered simultaneously or sequentially.

Specific embodiment 31. The method according to any one of specific embodiments 27-29, wherein the Type I IFN includes such as but not limited to IFNα-1, IFNα2 (e.g., IFNα-2a, IFNα-2b), IFNα4, IFNα5, IFNα6, IFNα7, IFNα8, IFNα10, IFNα13, IFNα14, IFNα16, IFNα17, IFNα21, IFNα-n1, IFNα-n3, IFNβ1 (e.g., IFNβ-1a, IFNβ-1b), and IFNβ3 either individually or in combinations of two or more thereof.

Specific embodiment 32. The method according to any one of specific embodiments 20-31, wherein the at least one therapeutic agent is selected from: one or more antibiotics, one or more anti-inflammatories, one or more antivirals, one or more anti-parasitic, and one or more mucoactive agents.

Specific embodiment 33. The method according to specific embodiment 32, wherein the one or more antibiotics is selected from: amoxicillin, azithromycin, erythromycin, penicillin, amoxicillin, and cefadroxil.

Specific embodiment 34. The method according to specific embodiment 32, wherein the one or more anti-inflammatories is selected from: chloroquine (e.g., 4-aminoquinoline), hydroxychloroquine (e.g., hydroxychloroquine sulfate), ibuprofen, naproxen, celecoxib, oxaprozin, piroxicam, aspirin (acetylsalicylic acid (ASA)), diclofenac, inhibitors of cyclo-oxygenase-1 (COX-1), COX-2, IL-10, IL-2, IL-6, IL-7, IL-8, IL-10, IL-12, tumor necrosis factor α (TNF-α), granulocyte-colony stimulating factor (G-CSF), interferon-γ-inducible protein (IP10), monocyte chemoattractant protein (MCP1), and macrophage inflammatory protein 1 alpha (MIP1A).

Specific embodiment 35. The method according to specific embodiment 32, wherein the one or more antivirals is selected from: chloroquine; 4-aminoquinoline; hydroxychloroquine (e.g., hydroxychloroquine sulfate); chlorpromazine; loperamide; lopinavir; lycorine; emetine; monensin sodium; mycophenolate mofetil; mycophenolic acid; phenazopyridine; pyrvinium pamoate; OYA1 (OyaGen, Inc.); remdesivir ((2S)-2-{(2R,3S,4R,5R)-[5-(4-Aminopyrrolo[2,1-f][1,2,4]triazin-7-yl)-5-cyano-3,4-dihydroxy-tetrahydro-furan-2-ylmethoxy]phenoxy-(S)-phosphorylamino}propionic acid 2-ethyl-butyl ester); neuraminidase inhibitor (e.g., oseltamivir, peramivir, zanamivir, laninamivir, 2,3-dehydro-2-deoxy-N-acetylneuraminic acid (DANA)); nucleoside analogs (acyclovir, cymevene, ribavirin (1-β-D-ribofuranosyl-1H-1,2,4-triazole-3-carboxamide)); favipiravir (T-705; 6-fluoro-3-hydroxy-2-pyrazinecarboxamide); protease inhibitor (e.g., indinavir, nelfinavir, saquinavir); reverse transcriptase inhibitor (e.g., lamivudine, zidovudine); amantadine; and foscarnet.

Specific embodiment 36. The method according to specific embodiment 32, wherein the one or more anti-parasitic is selected from: anti-malarials (e.g., chloroquine, hydroxychloroquine, amodiaquine and artesunate, atovaquone-proguanil, artemether-lumefantrine, quinine, parenteral quinine), anti-babesials (e.g., atovaquone, clindamycin-quinine), anti-amoebics (iodoquinol, paromomycin sulfate, diloxanide furoate, metronidazole, tinidazole, emetine), anti-giardials (e.g., metronidazole, tinidazole, furazolidone, albendazole), trypanocidals (e.g., nifurtimox, benznidazole, pentamidine, eflornithine, suramin, melarsoprol), anti-leishmanials (e.g., sodium stibogluconate, meglumine antimoniate, pentamidine, amphotericin B, paromomycin), anti-toxoplasma agents (e.g., sulfonamides: sulfadizine, sulfamethazine, sulfamerazine; and pyrimethamine), antipneumocystis agents (trimethoprim-sulfamethoxazole, pentamidine, trimethoprim-dapsone, clindamycin-primaquine), anti-trichomoniasis agents (e.g., metronidazole), anti-helminthics (e.g., vermicides, vermifuges, praziquantel, albendazole), anti-cestodals (praziquantel, niclosamide, albendazole), anti-nematodals (e.g., praziquantel, metrifonate, oxamniquine, bithionol, albendazole, diethylcarbamazine, ivermectin), and anti-scabietics (e.g., lindane, permethrin, benzyl benzoate, ivermectin, permethrin and oral ivermectin, topical ivermectin, synergized pyrethrins), and pediculicides (e.g., permethrin, pyrethrins, malathion, ivermectin)).

Specific embodiment 37. The method according to specific embodiment 32, wherein the one or more mucoactive agents is selected from: expectorants, mucolytics, mucokinetics, mucoregulators.

Specific embodiment 38. The method according to any one of specific embodiments 32, wherein the one or more mucoactive agents is selected from: guaifenesin, potassium iodide, acetylcysteine, sodium citrate, potassium citrate, Tolu balsam, vasaka, ammonium chloride, ambroxol, bromhexine, carbocisteine, erdosteine, mecysteine, and dornase alfa.

Specific embodiment 39. The method according to any one of specific embodiments 20-38, wherein IFN is administered in a pharmaceutical composition in an amount of 1 picogram (pg)/milliliter (mL)-100 micrograms (pg)/mL (e.g., 100 μg/mL-50 μg/mL, 1 nanogram (ng)/mL-1 μg/mL, 10 ng/mL-100 ng/mL).

Specific embodiment 40. The method according to any one of specific embodiments 20-39, wherein one or more of the therapeutic agents is administered in a pharmaceutical composition independently in an amount of 1 picogram (pg)/milliliter (mL)-100 micrograms (μg)/mL (e.g., 100 pg/mL-50 μg/mL, 1 nanogram (ng)/mL-1 μg/mL, 10 ng/mL-100 ng/mL).

Specific embodiment 41. The method according to any one of specific embodiments 22-40, wherein the aminoquinoline is administered in a pharmaceutical composition in an amount of 1 picogram (pg)/milliliter (mL)-100 micrograms (μg)/mL (e.g., 100 pg/mL-50 μg/mL, 1 nanogram (ng)/mL-1 μg/mL, 10 ng/mL-100 ng/mL).

Specific embodiment 42. The method according to any one of specific embodiments 22-41, wherein the corticosteroid is administered in a pharmaceutical composition in an amount of 1 picogram (pg)/milliliter (mL)-100 micrograms (μg)/mL (e.g., 100 pg/mL-50 μg/mL, 1 nanogram (ng)/mL-1 μg/mL, 10 ng/mL-100 ng/mL).

Specific embodiment 43. The method according to any one of specific embodiments 20-42, wherein the inhalable pharmaceutical composition formulation is selected from: an oral spray, a nasal spray, an aerosol, a liquid, a dry powder, a gas, or atomizable particles or droplets.

Specific embodiment 44. The method according to specific embodiment 43, wherein the spray, aerosol, liquid, dry powder, particles, or droplets have a particle size in a range to deposit said particles in the lungs, (e.g., have a particle size of from 0.5 microns to 5 microns as measured, for example, by dynamic light scattering).

Specific embodiment 45. The method according to any one of specific embodiments 20-44, wherein any administering step utilizes a nebulizer, inhaler (e.g., metered-dose inhalers, dry-powder inhalers), gas masks, or the like.

Specific embodiment 46. The method according to any one of specific embodiments 20-45, wherein the viral respiratory infection is caused by a virus selected from: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV).

Specific embodiment 47. The method according to specific embodiment 46, wherein the virus is a coronavirus selected from: human coronavirus 229E (HCoV-229E), HCoV-NL63, HCoV-OC43, HCoV HKU1, MERS-CoV, SARS-CoV, and SARS-CoV-2 or 2019-CoV.

Specific embodiment 48. The method according to any one of specific embodiments 20-47, wherein the viral respiratory infection has induced a disease, disorder, or condition in the subject selected from: a cold, bronchiolitis, croup, pneumonia, coronavirus disease 2019 (e.g., COVID-19), severe acute respiratory syndrome (SARS), middle East respiratory syndrome (MERS).

Specific embodiment 49. A method of treating COVID-19 in a subject in need thereof comprising administering an inhalable IFNα and/or inhalable IFNβ in a therapeutically effective amount; and administering azithromycin and/or hydroxychloroquine.

Specific embodiment 50. The method according to specific embodiment 49, wherein administrating the inhalable IFNα and/or inhalable IFNβ and administering azithromycin and/or hydroxychloroquine occurs simultaneously or sequentially.

Specific embodiment 51. A method of treating COVID-19 in a subject in need thereof comprising:
(a) administering an inhalable IFNα or IFNβ;
(b) administering inhalable azithromycin,
(c) administering inhalable hydroxychloroquine; and
(d) administering an inhalable corticosteroid; and
(e) optionally administering at least one therapeutic agent.

Specific embodiment 52. The method according to specific embodiment 51, wherein the inhalable IFNα, inhalable IFNβ, azithromycin, hydroxychloroquine, are administered simultaneously, sequentially, or in sequential combinations thereof.

Specific embodiment 53. A method of treating a viral respiratory infection in a subject in need thereof comprising administering to the subject an inhalable pharmaceutical composition comprising:
(a) at least one interferon (IFN);
(b) chloroquine or hydroxychloroquine, individually or in combination;
(c) a corticosteroid; and
(d) one or more pharmaceutically acceptable excipients, carriers, and/or diluents; and
(e) optionally administering at least one therapeutic agent.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A pharmaceutical composition comprising:
(a) at least one interferon (IFN);
(b) an aminoquinoline selected from chloroquine, hydroxychloroquine, and combinations thereof;
(c) a corticosteroid; and
(d) one or more pharmaceutically acceptable excipients, carriers, or diluents; and
(e) optionally at least one therapeutic agent.

2. The pharmaceutical composition according to claim 1, wherein the at least one interferon is a Type I IFN; a Type II IFN; and a Type III IFN, individually or with two or more in combination.

3. The pharmaceutical composition according to claim 1, wherein said at least one therapeutic agent is selected from: antibiotics, anti-inflammatories, antivirals, anti-parasitics, and mucoactive agents.

4. The pharmaceutical composition according to claim 1, wherein the pharmaceutical composition comprises 1 pg/mL-100 micrograms μg/mL of said inhalable interferon.

5. The pharmaceutical composition according to claim 1, wherein the pharmaceutical composition comprises 1 pg/mL-100 micrograms μg/mL of said aminoquinoline.

6. The pharmaceutical composition according to claim 1, wherein the pharmaceutical composition comprises 1 pg/mL-100 micrograms μg/mL of said inhalable corticosteroid.

7. The pharmaceutical composition according to claim 1, wherein the inhalable pharmaceutical composition formulation is selected from: an oral spray, a nasal spray, an aerosol, a liquid, a dry powder, a gas, or atomizable particles or droplets.

8. The pharmaceutical composition according to claim 22, wherein the spray, aerosol, liquid, dry powder, particles, or droplets have a particle size in a range of from 0.5 μm to 5 μm.

9. A method of treating a viral respiratory infection in a subject in need thereof comprising administering a therapeutically effective amount of the inhalable pharmaceutical composition according to claim 1.

10. A method of treating a viral respiratory infection in a subject in need thereof comprising:
(a) administering an interferon (IFN);
(b) administering an aminoquinoline selected from chloroquine, hydroxychloroquine, and combinations thereof;
(c) administering a corticosteroid.

11. The method according to claim 10, wherein the interferon, corticosteroid, chloroquine, and optional at least one therapeutic agent are each administered sequentially.

12. The method according to claim 10, wherein the interferon, corticosteroid, aminoquinoline, and optional at least one therapeutic agent are each administered simultaneously.

13. The method according to claim 10, wherein
(i) administration of the interferon and the corticosteroid is simultaneous by administration of a pharmaceutical composition comprising
(1) the interferon,
(2) the corticosteroid, and
(3) one or more pharmaceutically acceptable carriers, excipients, or diluents; and
(ii) the aminoquinoline is administered sequentially with respect to the pharmaceutical composition.

14. The method according to claim 10, wherein
(i) administration of the interferon and the aminoquinoline is simultaneous by administration of a pharmaceutical composition comprising:
(1) the interferon,
(2) the aminoquinoline, and
(3) one or more pharmaceutically acceptable carriers, excipients, or diluents; and
(ii) the corticosteroid is administered sequentially with respect to the pharmaceutical composition.

15. The method according to claim 10, wherein
(i) administration of the corticosteroid and the aminoquinoline is simultaneous by administration of a pharmaceutical composition comprising:
(1) the corticosteroid,
(2) the aminoquinoline, and
(3) one or more pharmaceutically acceptable carriers, excipients, or diluents;
(ii) the interferon is administered sequentially with respect to the pharmaceutical composition.

16. The method according to claim 10, further comprising:
(d) administering at least one therapeutic agent selected from one or more antibiotics, one or more anti-inflammatories, one or more antivirals, one or more anti-parasitic, and one or more mucoactive agents.

17. The method according to claim 10, wherein administering steps (a), (b), and (c), individually or in combinations of two or more thereof, utilizes a nebulizer, inhaler, or gas mask.

18. The method according to claim 10, wherein the viral respiratory infection is caused by a virus selected from influenza virus, respiratory syncytial virus, parainfluenza virus, adenovirus, rhinovirus, metapneumovirus, human metapneumovirus and endemic human coronaviruses, enterovirus, and coronavirus.

19. The method according to claim 38, wherein the viral respiratory infection is caused by coronavirus selected from human coronavirus 229E (HCoV-229E), HCoV-NL63, HCoV-OC43, HCoV HKU1, MERS-CoV, SARS-CoV, and SARS-CoV-2.

20. A method of treating COVID-19 in a subject in need thereof comprising:
(a) administering an inhalable IFNα or IFNβ;
(b) administering inhalable azithromycin,
(c) administering hydroxychloroquine; and
(d) administering an inhalable corticosteroid; and
(e) optionally administering at least one therapeutic agent.
